(19) United States
(12) Patent Application Publication
(10) Pub. No.: US 2016/0260046 A1
(43) Pub. Date: Sep. 8, 2016

(54) TRACKING WORKER ACTIVITY

(71) Applicant: Danqing CAI, Singapore (SG)

(72) Inventor: Danqing CAI, Singapore (SG)

(21) Appl. No.: 14/634,897

(22) Filed: Mar. 2, 2015

(51) Int. Cl. G06Q 10/06; A61B 5/11; A61B 5/024; A61B 5/01; G01K 13/02; A61B 5/00

(52) U.S. Cl. CPC G06Q 10/063114; G01K 13/02; A61B 5/681; A61B 5/02438; A61B 5/01; A61B 5/7278; A61B 5/1118; A61B 5/486; G01K 2013/024

(57) ABSTRACT

Disclosed herein are technologies for facilitating activity tracking In accordance with one aspect, sensor data is received from a wearable device. An activity type associated with a worker is recognized based on the sensor data. A fitness analysis may be performed, based on the sensor data and recognized activity type, to determine a stress level of the worker. One or more suggestions for improving the well-being of the worker may then be generated based on the stress level.

### TECHNICAL FIELD

The present disclosure relates generally to a framework for tracking worker activity.

### BACKGROUND

Warehouse workers face many challenges, including working 10 to 11 hour shifts, repetitive traveling and searching in large warehouses, operating along with hazardous machineries while trying to achieve high productivity goals. While it is critical for a logistics company to focus on improving warehouse efficiency so as to maintain the operating margin at a competitive level, it is also important to ensure their employees' work conditions are safe and sustainable.

One apparent way to address this issue is to replace manual labor with automated solutions, such as automated cranes, conveyors, etc., generally referred to as automated storage and retrieval systems (ASRS). With the help of automation, it is possible to achieve picking productivity up to 1,000 picks per person hour, or 1 pick every 3 seconds. This is, however, a costly solution that does not fit all businesses. According to some surveys, more than 80% of warehouses in Western Europe are still being operated manually. The number may be even higher in Asia where cost of labor is considerably lower.

### SUMMARY

A computer-implemented technology for facilitating activity tracking is described herein. In accordance with one aspect, sensor data is received from a wearable device. An activity type associated with a worker is recognized based on the sensor data. A fitness analysis may be performed, based on the sensor data and recognized activity type, to determine a stress level of the worker. One or more suggestions for improving the well-being of the worker may then be generated based on the stress level.

With these and other advantages and features that will become hereinafter apparent, further information may be obtained by reference to the following detailed description and appended claims, and to the figures attached hereto.

### BRIEF DESCRIPTION OF THE DRAWINGS

Some embodiments are illustrated in the accompanying figures, in which like reference numerals designate like parts, and wherein:

FIG. 1 is a block diagram illustrating an exemplary system;

FIG. 2 shows an exemplary data stream;

FIG. 3 shows an exemplary job workflow; and

FIG. 4 is a block diagram illustrating an exemplary method of tracking worker activity.

### DETAILED DESCRIPTION

In the following description, for purposes of explanation, specific numbers, materials and configurations are set forth in order to provide a thorough understanding of the present frameworks and methods and in order to meet statutory written description, enablement, and best-mode requirements. However, it will be apparent to one skilled in the art that the present frameworks and methods may be practiced without the specific exemplary details. In other instances, well-known features are omitted or simplified to clarify the description of the exemplary implementations of the present framework and methods, and to thereby better explain the present framework and methods. Furthermore, for ease of understanding, certain method steps are delineated as separate steps; however, these separately delineated steps should not be construed as necessarily order dependent in their performance.

The framework described herein may be implemented as a method, a computer-controlled apparatus, a computer process, a computing system, or as an article of manufacture such as a computer-usable medium.

One aspect of the present framework provides real-time tracking of activity via wearable devices. “Activity” as used herein generally refers to a specific physical action or state. By collecting real-time data on activities performed by, for example, warehouse workers, non-productive activities may be segregated from productive activities, jobs may be reassigned, realistic performance goals may be set, and signals suggesting non-compliance with safety standards or overtiredness may be detected, and so forth. Such features advantageously lead to higher operational efficiency and improves overall safety of workers or employees. These and various other features and advantages will be apparent from the following description.

For illustration purposes, the present framework may be described in the context of tracking activities of warehouse workers. It should be appreciated, however, that the present framework may also be applied in other types of applications, such as tracking activities of workers in a manufacturing environment, tracking activities associated with potential exposure to environmental hazards (e.g., high temperatures, etc.), and so forth.

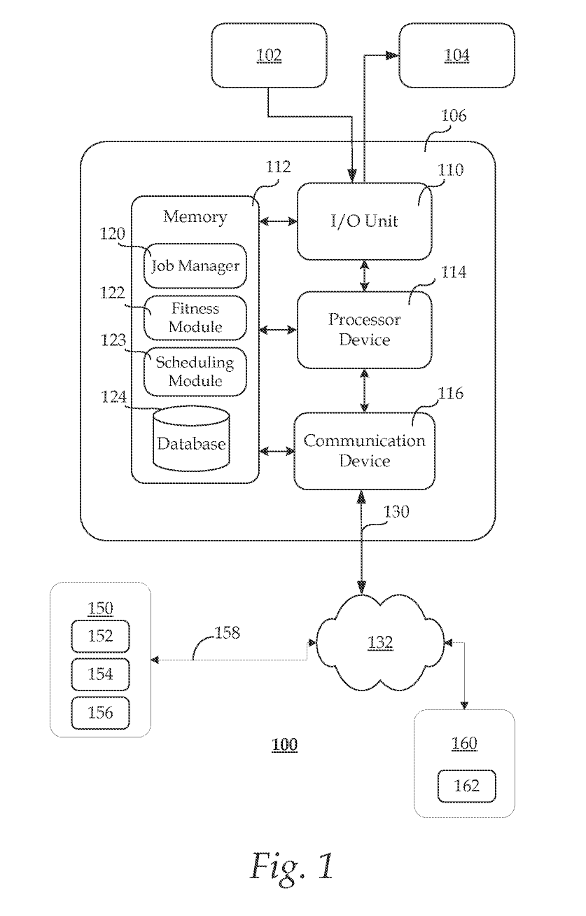

FIG. 1 shows a block diagram illustrating an exemplary system 100 that may be used to implement the framework described herein. System 100 includes a computer system 106 communicatively coupled to an input device 102 (e.g., keyboard, touchpad, microphone, camera, etc.) and an output device 104 (e.g., display device, monitor, printer, speaker, etc.). Computer system 106 may include a communications device 116 (e.g., a modem, wireless network adapter, etc.) for exchanging data with network 132 using a communications link 130 (e.g., telephone line, wireless or wired network link, cable network link, etc.). Network 132 may be a local area network (LAN) or a wide area network (WAN). The computer system 106 may be communicatively coupled to one or more wearable devices 150 and one or more computer systems 160 via network 132. For example, computer system 106 may act as a server and operate in a networked environment using logical connections to wearable devices 150 and computer systems 160.

Computer system 106 includes a processor device or central processing unit (CPU) 114, an input/output (I/O) unit 110, and a memory module 112. Other support circuits, such as a cache, a power supply, clock circuits and a communications bus, may also be included in computer system 106. In addition, any of the foregoing may be supplemented by, or incorporated in, application-specific integrated circuits. Examples of computer system 106 include a smart device (e.g., smart phone), a handheld device, a mobile device, a personal digital assistance (PDA), a workstation, a server, a portable laptop computer, another portable device, a mini-computer, a mainframe computer, a storage system, a dedicated digital appliance, a device, a component, other equipment, or some combination of these capable of responding to and executing instructions in a defined manner.

Memory module 112 may be any form of non-transitory computer-readable media, including, but not limited to, dynamic random access memory (DRAM), static random access memory (SRAM), Erasable Programmable Read-Only Memory (EPROM), Electrically Erasable Programmable Read-Only Memory (EEPROM), flash memory devices, magnetic disks, internal hard disks, removable disks, magneto-optical disks, Compact Disc Read-Only Memory (CD-ROM), any other volatile or non-volatile memory, or a combination thereof

Memory module 112 serves to store machine-executable instructions, data, and various programs, such as a job manager 120, a fitness module 122, a scheduling module 123 and a database (or data repository) 124 for implementing the techniques described herein, all of which may be processed by processor device 114. As such, the computer system 106 is a general-purpose computer system that becomes a specific purpose computer system when executing the machine-executable instructions. Alternatively, the various techniques described herein may be implemented as part of a software product. Each computer program may be implemented in a high-level procedural or object-oriented programming language (e.g., C, C++, Java, etc.), or in assembly or machine language if desired. The language may be a compiled or interpreted language. The machine-executable instructions are not intended to be limited to any particular programming language and implementation thereof. It will be appreciated that a variety of programming languages and coding thereof may be used to implement the teachings of the disclosure contained herein.

It should be appreciated that the different components of the computer system 106 may be located on different machines. For example, job manager 120, fitness module 122, scheduling module 123 and database 124 may reside in different physical machines. It should further be appreciated that the different components of wearable device 150 and computer system 160 may also be located in the computer system 106. All or portions of system 100 may be implemented as a plug-in or add-on.

Wearable device 150 and computer system 160 may include many components (e.g., processor device, communication device, memory, input and output devices, etc.) that are similar to computer system 106. Wearable device 150 and computer system 160 may include user interfaces 152 and 162 respectively to enable a user or worker to interact with computer system 106.

A wearable device 150 is an electronic device that is wearable or worn by a worker or user under, with or on top of clothing (e.g., smart watch, Google Glass, etc.). The wearable device 150 may be communicatively coupled to a smart device (e.g., smart phone, tablets, phablets, etc.) with computing capabilities via a wireless protocol (e.g., Bluetooth, NFC, WiFi, 3G, etc.). The wearable device 150 may include a user interface 152, a sensor module 154 and a user input device 156 for acquiring user input data. The user interface 152 may include one or more screens that display information (e.g., location on map, push notifications, etc.) generated based at least in part on sensor data received from the sensor module 154. The user input device 156 may include, for example, a touch screen, camera, image scanner, etc., that enables a user to manually input or scan information associated with a particular item (e.g., box, product, etc.) or job that the user is working on during a workflow. In addition, wearable device 150 may include a wireless communication device (not shown) that streams data 158 to and from computer system 106.

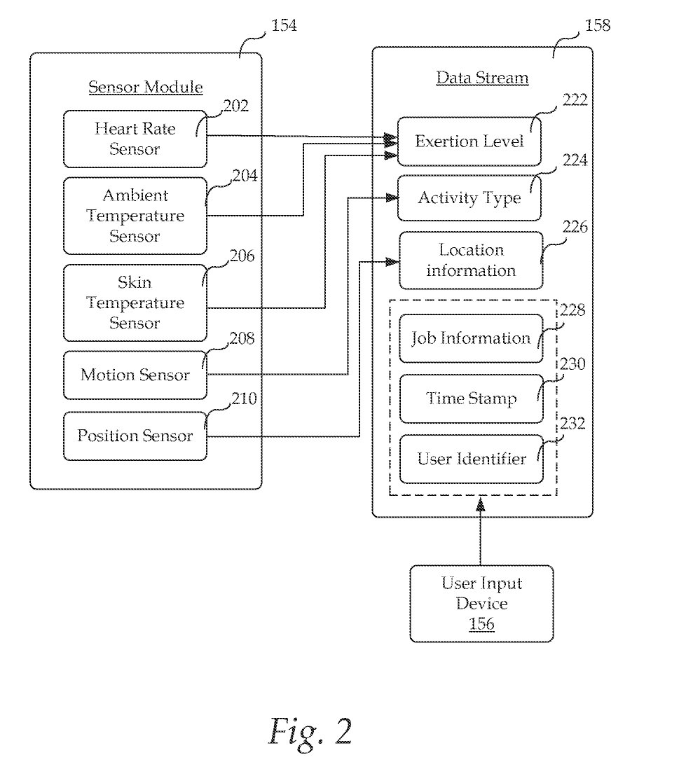

FIG. 2 shows an exemplary data stream 158 that may be generated based on sensor data from sensor module 154 and user input device 156 implemented in wearable device 150. It should be appreciated that the user input device 156 and the different components of the sensor module 154 may also be located on multiple wearable devices. In addition, one or more attributes of the data stream 158 may be generated by the wearable device 150 or the computer system 106.

In some implementations, sensor module 154 includes a heart rate sensor 202, an ambient temperature sensor 204, a skin temperature sensor 206, a motion sensor 208 and a position sensor 210. Other types of sensors, such as an oximetry sensor, may also be used. Some or all of the sensors may be integrated into one device. Heart rate sensor 202 is a personal monitoring device that measures the user's heart rate (or pulse) in real time. Ambient temperature sensor 204 collects information about the surrounding air temperature, while the skin temperature sensor 206 measures the user's skin surface temperature during a physical activity.

Motion sensor 208 captures information about the movement or orientation of the user that may be used to, for example, recognize gestures or activities. Motion sensor 208 may include, for example, an accelerometer, a gyroscope and/or electronic compass. Position sensor 210 may be any device that tracks the current location of the user. Position sensor 210 may either be an absolute position sensor or a relative sensor (or displacement sensor). An absolute position sensor includes, for example, a global positioning system (GPS) sensor. A relative sensor may be based on, for example, Bluetooth Low Energy (BLE), active Radio Frequency Identification (RFID), ultra wide band (UWB), and so forth.

Data stream 158 includes various types of attribute data (or values) that may be determined in part by the sensor data received from the sensor module 154 and user input data received from user input device 156. In some implementations, data stream 158 includes, but is not limited to, exertion level 222, activity type 224, location information 226, job information 228, time stamp 230 and user identifier 232. Other types of attributes may also be provided by the data stream 158. Wearable device 150 may include a processor device operative with computer-readable instructions or program code to determine values of the various attributes (e.g., exertion level). Alternatively, wearable device 150 may be communicatively coupled to a smart device (e.g., smart phone, tablets, phablets, etc.) with a processor device that executes computer-readable instructions to determine the various attribute values.

Exertion level 222 measures the use of physical or perceived energy. Exertion level 222 may be determined based on the heart rate, ambient temperature and/or skin temperature data acquired by the heart rate sensor 202, ambient temperature sensor 204 and skin temperature sensor 206. Exertion level 222 may include various predetermined levels, such as high, medium and low. High exertion level 222 may be determined in response to the heart rate and the skin temperature being above predetermined threshold values. Medium exertion level 222 may be determined in response to the heart rate being above a medium threshold level, the skin temperature being above a medium threshold level, or the ambient temperature being above a predetermined threshold level. Low exertion level 222 may be determined in response to none of these conditions being fulfilled.

Different activity types 224 may be recognized and categorized. For example, activity type may be “Idle”, “Travel” or “Work”. Each type of activity may be associated with sub-types. For example, in the context of warehouse work, the “Work” activity type may be associated with “Pick”, “Lift”, “Sort” or “Others” sub-types. It should be appreciated that other activity types or sub-types may also be defined. For example, other activity types may include “idling/resting”, “traveling by foot”, “traveling on forklift”, “loading/unloading by forklift”, “zone picking”, “not in warehouse”, and so forth.

Activity type 224 may be determined based at least in part on motion data, exertion level 222, location information 226 or a combination thereof. For instance, the “Idle” activity type may be determined in response to the exertion level 222 being low (i.e., falling below predetermined threshold value), motion data indicating there is no motion, and location information 226 indicating there is no change in location. The “Travel” activity type may be determined in response to the exertion level 222 being low or medium, motion data indicating there is no motion, and location information 226 indicating there is a change in location. The “Work” activity type may be determined in response to the exertion level 222 being medium or high (i.e., exceeding a predetermined threshold value), and motion data indicating there is acceleration or turning.

The sub-type of the “Work” activity type may be determined based on the motion data. For example, the sub-type “Pick” may be determined in response to the motion data indicating there is turning and the number of repetitions is less than a pre-determined number. The sub-type “Lift” may be determined in response to the motion data indicating there is acceleration. The sub-type “Sort” may be determined in response to the motion data indicating there is turning and the number of repetitions is more than a pre-determined number. The sub-type “Others” may be determined in response to none of these conditions being fulfilled.

Location information 226 may be determined based on positioning data from position sensor. Location information 226 may indicate the current location of the worker, and/or the distance travelled by the worker. The current location of the worker may be an absolute or relative indoor location with an accuracy range within, for instance, centimeters.

User input device 156 may be used to provide job information 228, time stamp 232 of the job, and identification information of the user or worker 232 (e.g., name, unique employee number, etc.). Job information 228 may include information (e.g., identification, type, status, etc.) associated with the job and items handled during the job workflow.

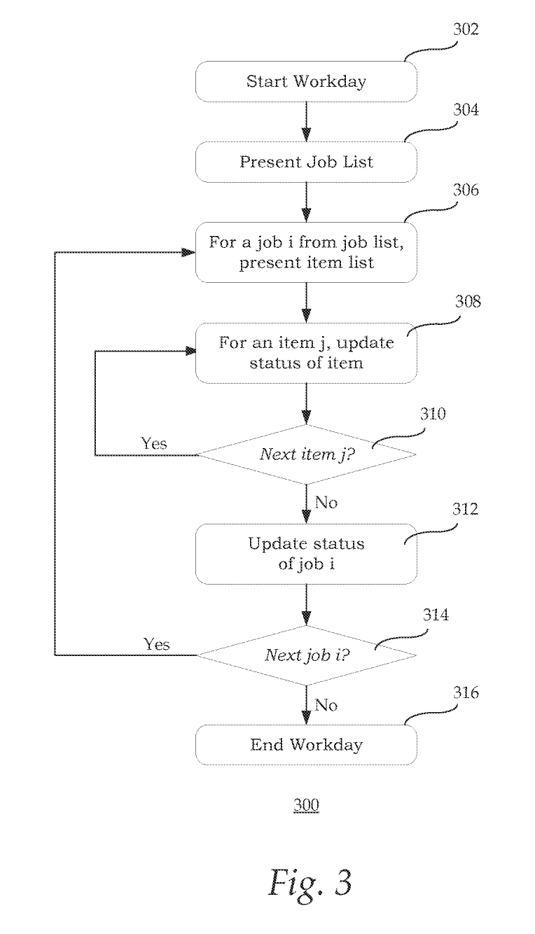

FIG. 3 shows an exemplary job workflow 300. The job workflow 300 may be performed by, for example, a warehouse worker who is responsible for managing items stored in a warehouse. Other types of activities may also be performed.

At 302, the worker starts his or her workday. The worker may be equipped with, for example, a wearable device 150. As discussed previously, the wearable device 150 may include a user interface 152, a sensor module 154 and a user input device 156. The sensor module 154 may continuously acquire sensor data and generate one or more current attributes (e.g., exertion level 222, activity type 224, location information 226, etc.) in the data stream 158 during the entire workflow 300, as previously described. Such attributes may be displayed to the worker via the user interface 152 at any time during the workflow 300 or in response to the user's actions.

At 304, the user interface 152 presents a job list. The job list may be retrieved from, for example, job manager 120 or computer system 106. The job list includes information of one or more N jobs assigned to the worker, wherein N is an integer representing the total number of jobs. Each job may be associated with job identification information (e.g., name, unique identifier, etc.), type (e.g., pick, load, unload, replenish, sort, combination thereof, etc.) and status information (e.g., “New”, “Started”, “Completed”, “Cancelled”, etc.).

At 306, the user interface 152 presents an item list associated with a job i from the job list, wherein i denotes a job index from 1 to N. The item list includes information of one or more M items, wherein M is an integer representing the total number of items. Each item may be associated with item identification information (e.g., name, unique identifier, location, etc.), type (e.g., bulk, high rack, shelf, open, etc.), status information (e.g., “Available”, “Out of stock”, etc.), and so forth.

At 308, status information associated with an item j on the item list is updated. In some implementations, the status information is updated via a user input device 156. For example, the user input device 156 includes a scanner that enables the user to scan an image or barcode of the item. Alternatively, the user input device 156 may include a voice recognition module that recognizes words based on the worker's speech. As another alternative, the user input device 156 may include a touchscreen that enables a worker to input updated status information. Other types of user input devices may also be used.

At 310, the wearable device 150 determines if there is another item j on the item list that needs to be processed. If so, the process 300 returns to step 308. If all the items on the item list has already been processed, the process 300 continues to step 312.

At 312, status information associated with the job i on the job list is updated. In some implementations, the status information is updated via a user input device 156. For example, the user input device 156 includes a scanner that enables the user to scan an image associated with the job status. Alternatively, the user input device 156 may include a voice recognition module that recognizes words based on the worker's speech. As another alternative, the user input device 156 may include a touchscreen that enables a worker to input updated status information. Other types of user input devices may also be used.

At 314, the wearable device 150 determines if there is another job i on the job list that needs to be processed. If so, the process 300 returns to step 306. If all the jobs on the job list has already been processed, the process 300 continues to step 316. At 316, the workday ends.

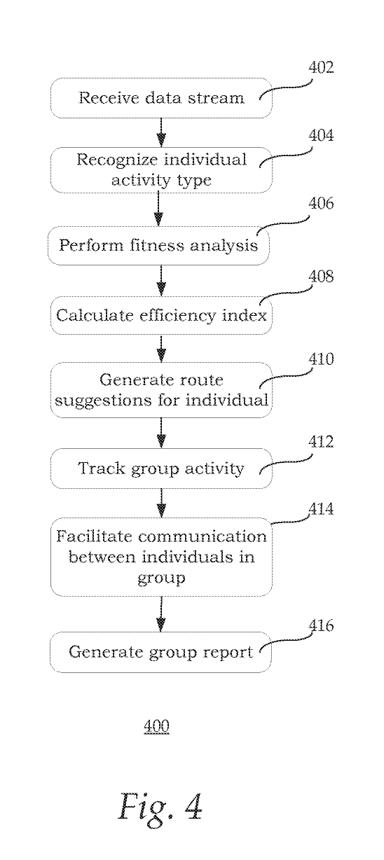

FIG. 4 is a block diagram illustrating an exemplary method 400 of tracking worker activity. The computer system 106 of FIG. 1 may be configured by computer program code to implement some or all acts of the process 400. In some implementations, the wearable device 150 may also be configured by computer program code to implement some or all acts of the process 400. While process flow 400 describes a series of acts that are performed in sequence, it is to be understood that process 400 is not limited by the order of the sequence. For instance, some acts may occur in a different order than that described. In addition, an act may occur concurrently with another act. In some instances, not all acts may be performed.

At 402, computer system 106 receives the data from the wearable device 150. The data may include the sensor data from the sensor module 154. Alternatively, or additionally, the data may include one or more attributes of data stream 158 that are generated while the user (or worker) performs a job workflow (e.g., workflow 300 of FIG. 3).

At 404, job manager 120 recognizes individual activity type to track the individual activity of the worker. Such tracking may be initiated in response to the individual activity mode being activated via, for example, user interface 152. The individual activity mode may be activated, for example, in response to the individual worker logging into the tracking application at the start of the workday. The activity of the individual worker may be tracked by recognizing the activity type 224 based on sensor data, such as motion data from motion sensor 208, exertion level 222 and location information 226, as described previously.

At 406, fitness module 122 performs fitness analysis. The fitness analysis may be performed by detecting the worker's stress level based on, for example, exertion level 222 and/or activity type 224. The fitness analysis may also include determining the total distance traveled by the worker based on location information 226 and calories consumption based on the distance traveled and activity type 224. Based on the fitness analysis results, fitness module 122 may generate suggestions to improve the well-being of the individual worker. For example, fitness module 122 may generate and initiate display of a notification, via user interface 152, to remind the worker to take a rest from the job in response to the stress level being high (or above a pre-determined threshold).

At 408, job manager 120 calculates an individual efficiency index based at least in part on the recognized activity types 224 and/or job information 228. In some implementations, computer system 106 summarizes the time spent for each trip, each job, each shift, etc. Computer system 106 may classify the recognized activity types into productive and non-productive activities. A productive activity advances the fulfillment of a job or function, while a non-productive activity does not advance the fulfillment of a job or function. For example, a “Work” activity type is a productive activity, while an “Idle” activity type is a non-productive activity. An efficiency index may be determined based on the productive time spent by the individual worker on productive activities. The efficiency index may be determined by, for example, calculating the ratio (or percentage) of productive time to total time spent on the job by the worker. For example, an efficiency index of 60% indicates that 60% of the worker's time is categorized as productive.

At 410, scheduling module 123 generates one or more suggestions of routes for the individual worker to travel to a location of an item for processing. Scheduling module 123 may initiate, via user interface 152, generation of a visualization or display image (e.g., map) of the locations of the remaining items to process by the worker, as extracted from job information 228. Scheduling module 123 may also indicate in the visualization where real-time hotspots are located. Such hotspots may indicate, for example, locations of potential inefficiency or workers with low efficiency indices (i.e., below a predetermined threshold value) that may be minimized by job re-assignment. The hotspots may also indicate storage locations that are in high demand that are to be avoided.

Additionally, scheduling module 123 may generate real-time suggestions of routes to the remaining items to be processed so as to improve the work efficiency of the workers as a whole. The suggested routes may also be indicated in the visualization. Each route may be determined based on the location information 226 and job information 228. For example, the scheduling module 123 may first extract the current location of the worker and the location of the item to be processed from the location information 226 and the job information 228. The route may then be determined based on, for example, the shortest distance between the current location of the worker and the location of the item.

At 412, job manager 120 tracks group activity. Such tracking may be initiated in response to the group activity mode being activated via, for example, user interface 162. The group activity mode may be activated in response to a user selection at user interface 162. To track the activities of a group of workers, the job manager 120 collects and analyzes sensor data or multiple data streams from different wearable devices 150 associated with different workers. Activities performed by the different workers may be recognized based on the multiple data streams.

In some implementations, job manager 120 determines a group level efficiency index based on the recognized activities. Job manager 120 may classify the recognized activities into productive and non-productive activities, and determine the group level efficiency index based on productive time spent by all workers on productive activities. The group level efficiency index may be determined by, for example, calculating the ratio (or percentage) of productive time of all workers to total time spent on the jobs by all workers.

Jobs may be re-assigned if necessary. In some implementations, jobs are re-assigned based on occurrence of ad hoc (or unplanned) events, such as prolonged delay in a particular job. The job re-assignment may be triggered manually by, for example, a co-worker volunteering to take over the job.

At 414, while in the group activity tracking mode, job manager 120 facilitates communication between individual workers within the group. In some implementations, job manager 120 shares, via the user interface 152, information of locations or traveling paths to the next locations of other individual workers. Workers may also exchange messages (e.g., text or voice messages) via the user interface 152.

At 416, job manager 120 generates a group report. The group report may be presented via the user interfaces 152 or 162. In some implementations, the group report presents information of one or more current attributes (e.g., “Activity Type” 224, “Exertion Level” 222, “Location Information” 226, etc.) of all the workers in the group. The group report may also include a daily summary of the attribute information at particular times of the day. Other information, such as the group level efficiency index, may also be presented in the group report.

Although the one or more above-described implementations have been described in language specific to structural features and/or methodological steps, it is to be understood that other implementations may be practiced without the specific features or steps described. Rather, the specific features and steps are disclosed as preferred forms of one or more implementations.

1. A device wearable by a worker, comprising:
a heart rate sensor that measures the worker's heart rate;
an ambient temperature sensor that measures ambient temperature of air surrounding the worker;
a skin temperature sensor that measures skin temperature of the worker;
a processor device operative with computer-readable program code to perform one or more steps including determining an exertion level of the worker based on the heart rate, the ambient temperature and the skin temperature; and
a user interface that displays a notification that reminds the worker to rest in response to the exertion level exceeding a predetermined threshold value.

2. The device of claim 1 further comprising a motion sensor that captures movement information of the worker, and wherein the processor device is further operative with the computer-readable code to determine an activity type based on the movement information.

3. The device of claim 2 wherein the activity type comprises “Idle”, “Travel” or “Work”.

4. The device of claim 1 further comprising a position sensor that tracks a current location of the worker, and wherein the processor device is further operative with the computer-readable code to determine location information based on the current location of the worker.

5. The device of claim 1 wherein the user interface presents a job list retrieved from a computer system, wherein the job list comprises status information of one or more jobs assigned to the worker.

6. The device of claim 5 further comprising a user input device that enables the worker to update the status information of the one or more jobs.

7. The device of claim 5 wherein the user interface presents an item list associated with a job on the job list, wherein the item list comprises status information of one or more items on the item list.

8. The device of claim 7 further comprising a user input device that enables the worker to update the status information of the one or more items.

9. A system for tracking worker activity, comprising:
a non-transitory memory device for storing computer readable program code; and
a processor in communication with the memory device, the processor being operative with the computer readable program code to perform steps including:
receiving sensor data from at least one wearable device associated with a worker,
recognizing, based on at least the sensor data, an activity type associated with the worker,
performing, based on the sensor data and the recognized activity type, a fitness analysis that determines a stress level of the worker, and
generating, based on the stress level, one or more suggestions to improve well-being of the worker.

10. The system of claim 9 wherein the sensor data comprises a heart rate, an ambient temperature, a skin temperature, or a combination thereof, and the processor is operative with the computer readable program code to determine an exertion level of the worker based on the heart rate, the ambient temperature, the skin temperature, or a combination thereof.

11. The system of claim 10 wherein the processor is operative with the computer readable program code to recognize the activity type based at least in part on the exertion level.

12. The system of claim 9 wherein the processor is operative with the computer readable program code to recognize the activity type based at least in part on motion data, exertion level, location information or a combination thereof.

13. The system of claim 12 wherein the processor is operative with the computer readable program code to recognize the activity type as an “Idle” activity type in response to the exertion level falling below a predetermined threshold value, the motion data indicating no motion and the location information indicating no change in location.

14. The system of claim 12 wherein the processor is operative with the computer readable program code to recognize the activity type as a “Travel” activity type in response to the exertion level falling below a predetermined threshold value, the motion data indicating no motion and the location information indicating a change in location.

15. The system of claim 9 wherein the one or more suggestions comprises a reminder for the worker to take a rest from a job.

16. The system of claim 9 wherein the processor is operative with the computer readable program code to classify the recognized activity type into a productive or non-productive activity, and to determine an individual efficiency index based on productive time spent by the worker on productive activities on a job.

17. The system of claim 9 wherein the processor is operative with the computer readable program code to generate one or more suggestions of routes to a location of an item for processing by the worker.

18. The system of claim 9 wherein the processor is operative with the computer readable program code to track group activity by recognizing activity types associated with multiple workers.

19. The system of claim 18 wherein the processor is operative with the computer readable program code to classify the recognized activity types into productive or non-productive activities, and to determine a group level efficiency index based on productive time spent by the multiple workers on productive activities on a job.

20. A non-transitory computer readable medium embodying a program of instructions executable by machine to perform steps for tracking worker activity comprising:
receiving sensor data from at least one wearable device associated with a worker;
recognizing, based on at least the sensor data, an activity type associated with the worker;
performing, based on the sensor data and the recognized activity type, a fitness analysis that determines a stress level of the worker; and
generating, based on the stress level, one or more suggestions to improve well-being of the worker.
